(19) United States
(12) Patent Application Publication
(10) Pub. No.: US 2008/0255766 A1
(43) Pub. Date: Oct. 16, 2008

(54) Methods and kits for the diagnosis of sickle cell

(75) Inventors: Michael Spain, Austin, TX (US); James P. Mapes, Austin, TX (US); Ralph L. McDade, Austin, TX (US); Kenneth Pass, Glenmont, NY (US)

Correspondence Address: FOLEY AND LARDNER LLP;SUITE 500, 3000 K STREET NW, WASHINGTON, DC, 20007, US

(73) Assignees: Rules-Based Medicine, Inc.; Health Research Inc.

(21) Appl. No.: 12/068,518

(22) Filed: Feb. 7, 2008

Related U.S. Application Data
(60) Provisional application No. 60/890,305, filed on Feb. 16, 2007.

(51) Int. Cl. G01N 33/53; C12Q 1/00

(52) U.S. Cl. 702/19; 435/4; 435/7.1

(57) ABSTRACT

Provided are methods for the detection and diagnosis of sickle cell. The methods are based on the discovery that abnormal levels of selected analytes in sample fluid, typically blood samples, of patients who are at risk are supportive of a diagnosis of sickle cell. At least two new biomarkers for sickle cell are thus disclosed, Eotaxin and Monocyte Chemotactic Protein-1. Altogether the concentrations of eleven analytes provide a sensitive and selective picture of the patient's condition, namely, whether the patient is suffering from sickle cell. Other important biomarkers for sickle cell are described, including but not limited to IL-12p40, SHBG, MMP-9, Adiponectin, Haptoglobin, FGF basic, IgM, Growth Hormone, Factor VII. Kits containing reagents to assist in the analysis of fluid samples are also described.

### CROSS REFERENCE TO RELATED APPLICATIONS

This application claims priority to U.S. provisional application No. 60/890,305 filed Feb. 16, 2007, the disclosure of which is incorporated herein by reference in its entirety.

### BACKGROUND

1. Field of the Invention

The invention generally relates to methods, kits and reagents for detection and/or diagnosis of sickle cell disease.

2. Description of the Related Art

Sickle Cell Disease (SCD) is caused by the malfunction of the red blood cells in affected individuals causing a very severe form of anemia. The gene defect for sickle cell disease is an autosomal recessive genetic trait and is unknowingly passed down from generation to generation. This “faulty” gene usually emerges when two carrier parents pass the gene to their offspring. For each pregnancy of two such carriers, there is a 25% chance that the child will be born with the disease and a 50% chance the child will be a carrier for the gene defect.

It is estimated that on average, sickle cell disease affects one of every 1,300 infants in the general population and approximately one of every 400 of African descent. Though seemingly more prevalent in individuals of African and Mediterranean ancestry, the disease is not limited to these races. Hence, individuals should not forgo screening for this disorder based on race criteria alone. These statistics provide the primary rationale for improving the screening and diagnosis of patients at risk for sickle cell.

SCD can arise in children of all backgrounds, and there is an extremely high mortality for sufferers under the age of five. Although better treatments are becoming available to help cope with this disease, currently, there is no cure for sickle cell disease. Rather, SCD is treated with folic acid and penicillin throughout the life of an affected individual. Untreated newborns often develop septicemia, an infection of the blood, and die within a few weeks of birth.

The selectivity and sensitivity of current assays for sickle cell are lacking, with the frequency of false positives and false negatives at an undesirable level. Thus, there is a critical need to develop additional biomarkers for early detection of sickle cell.

### SUMMARY OF THE INVENTION

A method for rapid detection and/or accurate diagnosis of sickle cell is provided. The method can be practiced with a determination of the concentrations of one, two or more biomarkers in a patient fluid sample. Depressed (or elevated, as the case might be) levels of the one or two biomarkers, which are statistically different from levels found in “normals” (that is, control subjects not suffering from sickle cell), support a positive diagnosis of sickle cell. Preferably, the method utilizes a panel of analytes or “biomarkers,” up to twelve or more substances found in a sample fluid (e.g., whole blood, serum, plasma, or urine), to help support a positive or negative diagnosis of sickle cell. Up to 99% accuracy in making a correct diagnosis is provided by the method.

According to the invention a method of diagnosing sickle cell in a human subject, preferably a newborn, suspected of suffering from sickle cell is provided, which comprises: (a) obtaining a fluid sample (b) determining the concentration of Eotaxin in said fluid sample; (c) deciding if the determined concentration of Eotaxin in said fluid sample is statistically different from that found in a control group of human subjects, whereby a statistically different depressed concentration of Eotaxin supports a positive diagnosis of sickle cell. Any one of a number of fluid samples can be tested. Preferably, the fluid sample is selected from whole blood, plasma, blood spots, serum, or urine. It has been discovered that a measured concentration of about 300 pg/mL or less of Eotaxin in the fluid sample supports a positive diagnosis of sickle cell.

In another aspect of the invention a method is provided for diagnosing sickle cell in a human subject, preferably a newborn, suspected of suffering from sickle cell, which method comprises: (a) obtaining a fluid sample from a human subject (b) determining the concentration of Monocyte Chemotactic Protein-1 in said fluid sample; (c) deciding if the determined concentration of Monocyte Chemotactic Protein-1 in said fluid sample is statistically different from that found in a control group of human subjects, whereby a statistically different depressed concentration of Monocyte Chemotactic Protein-1 supports a positive diagnosis of sickle cell. It has been found that a measured concentration of about 2000 pg/mL or less of Monocyte Chemotactic Protein-1 in said fluid sample supports a positive diagnosis of sickle cell.

Still another aspect of the invention relates to a method of diagnosing sickle cell in a human subject suspected of suffering from sickle cell, comprising: (a) obtaining a fluid sample from a human subject suspected of suffering from sickle cell; (b) determining the concentrations of Eotaxin and Monocyte Chemotactic Protein-1 in said fluid sample; (c) deciding if the determined concentrations of Eotaxin and Monocyte Chemotactic Protein-1 in said fluid sample are statistically different from that found in a control group of human subjects, whereby a statistically different depressed concentration of Eotaxin and a statistically different depressed concentration of Monocyte Chemotactic Protein-1 together support a positive diagnosis of sickle cell.

In a preferred embodiment, the method of the invention further comprises determining the concentration in said fluid sample of at least one of Eotaxin, MCP-1, Interleukin-12p40 (IL-12p40), Sex Hormone Binding Globulin (SHBG), Matrix Metalloproteinase-9 (MMP-9), Adiponectin, Haptoglobin, Fibroblast Growth Factor basic (FGF basic), Immunoglobulin M (IgM), Growth Hormone, Factor VII, or any combination thereof. It has been found that statistically different depressed concentrations, compared to control levels, of all analytes mentioned above except Eotaxin, support a positive diagnosis of sickle cell. In particular, certain threshold levels of analytes in the sample fluids have been found to be important in the detection or diagnosis of sickle cell, IL-12p40, SHBG, MMP-9, Adiponectin, Haptoglobin, FGF basic, IgM, Growth Hormone, Factor VII.

Various techniques for assessing the importance of certain biomarkers in arriving at a diagnosis is also described herein. One such technique is a projection of compiled results on a proximity map, whereby the proximity of a subject's determined concentrations to a cluster of other subjects' determined concentrations, who were previously diagnosed as having suffered from sickle cell, contributes to a positive diagnosis of sickle cell. Other techniques include the application of one or more statistical methods (e.g., linear regression analysis, classification tree analysis, heuristic nave Bayes analysis and the like).

Also provided is a kit comprising reagents for determining the concentration in a fluid sample of a panel of analytes including Eotaxin, Monocyte Chemotactic Protein-1 and one or more of IL-12p40, SHBG, MMP-9, Adiponectin, Haptoglobin, FGF basic, IgM, Growth Hormone, Factor VII. The reagents may include antibodies against the members of a given panel of analytes. Furthermore, the reagent may be immobilized on a substrate, which substrate may comprise a two-dimensional array, a microtiter plate, or multiple bead sets.

The methods may further comprise comparing the levels of the one, two, or more biomarkers in a patient's blood with levels of the same biomarkers in one or more control samples by applying a statistical method such as: linear regression analysis, classification tree analysis and heuristic nave Bayes analysis. The statistical method may be, and typically is performed by a computer process, such as by commercially available statistical analysis software. In one embodiment, the statistical method is a classification tree analysis, for example CART (Classification and Regression Tree). Results for a particular patient or subject, whose sample fluid is tested against a panel of biomarkers according to the method, can be projected onto a proximity map. The proximity of a particular patient's biomarker concentration results to one of at least two populations (those previously diagnosed as having suffered from sickle cell and normals) supports a either a positive or negative diagnosis of ACS.

An article of manufacture is provided which comprises binding reagents specific for at least one of Eotaxin and Monocyte Chemotactic Protein-1, preferably both biomarkers. More preferably, a kit is provided which comprises binding reagents specific for Eotaxin, MCP-1, IL-12p40, SHBG, MMP-9, Adiponectin, Haptoglobin, FGF basic, IgM, Growth Hormone, Factor VII. In a preferred embodiment, each binding reagent is immobilized on a substrate. For example, monoclonal antibodies against Eotaxin, Monocyte Chemotactic Protein-1 and the other biomarkers described herein are immobilized independently to one or more discrete locations on one or more surfaces of one or more substrates. The substrates may be beads comprising an identifiable biomarker, wherein each binding reagent is attached to a bead comprising a different identifiable biomarker than beads to which a different binding reagent is attached. The identifiable biomarker may comprise a fluorescent compound, a quantum dot, or the like.

In another embodiment, a method is provided for determining the occurrence of sickle cell in a patient, comprising determining levels of at least one of Eotaxin and Monocyte Chemotactic Protein-1.

Other aspects of the invention will become apparent to those of ordinary skill after considering the detailed descriptions provided herewith.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

FIG. 1 is a projection of a proximity map (P<0.05) using principal component analysis (PCA), a technique for simplifying a dataset, by reducing multidimensional datasets to lower dimensions for analysis. PCA linearly transforms data to a new coordinate system such that the greatest variance by any projection of the data comes to lie on the first coordinate (called the first principal component), the second greatest variance on the second coordinate, and so on. PCA can be used for dimensionality reduction in a dataset while retaining those characteristics of the dataset that contribute most to its variance, by keeping lower-order principal components and ignoring higher-order ones. Such low-order components often contain the “most important” aspects of the data. But this is not necessarily the case and depends on the application.

FIG. 2 is another projection of a proximity map using principal component analysis (P<0.01).

### DETAILED DESCRIPTION OF THE PREFERRED EMBODIMENTS

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Provided herein is a multifactorial assay for rapid identification of a patient suffering from sickle cell. Identified below are certain sample fluid (e.g., blood) analytes or biomarkers useful in the detection and/or diagnosis of sickle cell. It has been found that Monocyte Chemotactic Protein-1 (MCP-1) has been found to be under-expressed in patients suffering from or who have suffered sickle cell abnormalities. Also identified as being useful in the detection or proper diagnosis of subjects suffering from sickle cell are the biomarkers Eotaxin, Interleukin-12p40 (IL-12p40), Sex Hormone Binding Globulin (SHBG), Matrix Metalloproteinase-9 (MMP-9), Adiponectin, Haptoglobin, Fibroblast Growth Factor basic (FGF basic), Immunoglobulin M (IgM), Growth Hormone, Factor VII.

The parameters for establishing the significance of one or more biomarkers for the diagnosis of sickle cell are determined statistically by comparing normal or control blood (preferably, e.g., serum or plasma) levels of these biomarkers with blood levels in patients clinically and properly diagnosed as having sickle cell. The statistical data presented below in Table 1 identify certain mean values and accompanying standard deviations for the blood levels of the above-described biomarkers in sickle cell patients and in normals.

TABLE 1
Significant Analytes in SCD Study

| | | | | Control | | Control |
|---|---|---|---|---|---|---|
| | | | Sickle | v SS | | v Carrier |
| | Units | Control | Cell | t-test | Carrier | t-test |
| Eotaxin | pg/mL | 393 | 165 | 7.70E−06 | 163 | 2.40E−05 |
| IL-12p40 | ng/mL | 0.92 | 0.5 | 2.20E−03 | 0.9 | 7.30E−01 |
| SHBG | nmol/L | 28 | 19 | 8.70E−03 | 23 | 3.10E−01 |
| MMP-9 | ng/mL | 125 | 254 | 9.10E−03 | 97 | 1.40E−01 |
| Adiponectin | ug/mL | 12 | 8.7 | 1.20E−02 | 11 | 6.80E−01 |
| Haptoglobin | mg/mL | 0.027 | 0.02 | 1.40E−02 | 0.023 | 8.40E−02 |
| FGF basic | pg/mL | 265 | 124 | 1.80E−02 | 116 | 8.40E−03 |
| MCP-1 | pg/mL | 2401 | 1369 | 1.90E−02 | 764 | 7.80E−07 |
| IgM | mg/mL | 0.062 | 0.037 | 2.20E−02 | 0.059 | 8.00E−01 |
| Growth | ng/mL | 18 | 31 | 2.60E−02 | 22 | 4.40E−01 |
| Hormone | | | | | | |
| Factor VII | ng/mL | 169 | 114 | 3.60E−02 | 150 | 5.50E−01 |

It is understood that these values are approximate. Statistical methods can be used to define the critical range of values. Typically within one standard deviation of those approximate values might be considered as statistically significant values for determining a statistically significant difference, preferably two standard deviations. For this reason, the word “about” is used in connection with the stated values. “Statistical classification methods” are used to identify biomarkers capable of discriminating normal patients from patients with sickle cell and are further used to determine critical blood values for each biomarker for discriminating between such patients. Certain statistical methods can be used to identify discriminating biomarkers and panels thereof. These statistical methods may include, but are not limited to: 1) linear regression; 2) classification tree methods; and 3) statistical machine learning to optimize the unbiased performance of algorithms for making predictions. Each of these statistical methods is well-known to those of ordinary skill in the field of biostatistics and can be performed as a process in a computer. A large number of software products are available commercially to implement statistical methods, such as, without limitation, S-PLUS™, commercially available from Insightful Corporation of Seattle, Wash.

The invention is based on an evaluation of at least Eotaxin levels, alone or in combination with levels of immunological Monocyte Chemotactic Protein-1 and/or other biomarkers listed in Table 1, in serum for diagnosis of sickle cell in all stages of it progression. The invention is also based on the evaluation of at least immunological Monocyte Chemotactic Protein-1 levels, optionally in combination with levels of at least Eotaxin. Patients with sickle cell are at considerable risk for serious complications, and outcomes can be improved with appropriate diagnosis and therapy. Thus, rapid and accurate diagnosis is critical for patient care.

The results described herein demonstrate that serum Eotaxin levels are depressed in sickle cell patients. Thus, Eotaxin can be used as an early biomarker of sickle cell conditions. By the same token, Monocyte Chemotactic Protein-1 levels are depressed in sickle cell conditions. Thus, Monocyte Chemotactic Protein-1 can be used as an early biomarker of sickle cell conditions.

The present method includes measuring the level of Eotaxin and/or Monocyte Chemotactic Protein-1 in a biological sample (e.g., whole blood, plasma, serum, blood spots or urine and the like) from a patient; comparing the respective levels with that of control subjects; and diagnosing the state of disease based on the level of Eotaxin or Monocyte Chemotactic Protein-1 relative to that of control (i.e., normal) subjects. A patient can be diagnosed with sickle cell abnormalities if the level of Eotaxin and/or Monocyte Chemotactic Protein-1 is decreased relative to controls.

A typical control value for Eotaxin is in the range of about 300-600 pg/mL. A concentration of about 300 pg/mL or less in a patient sample supports a positive diagnosis of SCD. The general range for depressed SCD values of Eotaxin is about 100-300 pg/mL.

A typical control value for Monocyte Chemotactic Protein-1 is in the range of about 2000-3000 pg/mL. An immunological concentration of about 2000 pg/mL or less in a patient sample supports a positive diagnosis of SCD. The general range for depressed values of immunological Monocyte Chemotactic Protein-1 concentration is about 1000-2000 pg/mL.

Eotaxin and Monocyte Chemotactic Protein-1 can be captured with anti-Eotaxin and anti-Monocyte Chemotactic Protein-1 polyclonal antibodies, respectively, or with corresponding monoclonal antibodies. The diagnostic method may also include measuring the levels of one or more additional analytes selected from the group consisting of: IL-12p40, SHBG, MMP-9, Adiponectin, Haptoglobin, FGF basic, IgM, Growth Hormone, Factor VII, and diagnosing the patient's condition based on the level of the additional analyte and the level of Eotaxin and/or Monocyte Chemotactic Protein-1 relative to that of control subjects.

Analyte levels can be measured using an immunoassay such as an ELISA or a multiplexed method as described below, and in more detail by Chandler et al., U.S. Pat. No. 5,981,180 (Luminex Corporation).

The analytes used in the method of the invention can be detected, for example, by a binding assay. For example, a sandwich immunoassay can be performed by capturing Eotaxin and Monocyte Chemotactic Protein-1 from a biological sample with antibodies having specific binding affinity for each protein, which then can be detected with a labeled antibody having specific binding affinity for each analyte. Alternatively, standard immunohistochemical techniques can be used to detect Eotaxin and Monocyte Chemotactic Protein-1 using such antibodies. Antibodies having affinity for Eotaxin and Monocyte Chemotactic Protein-1 are available.

The term “binding reagent” and like terms, refers to any compound, composition or molecule capable of specifically or substantially specifically (that is with limited cross-reactivity) binding another compound or molecule, which, in the case of immune-recognition is an epitope. The binding reagents typically are antibodies, preferably monoclonal antibodies, or derivatives or analogs thereof, but also include, without limitation: Fᵥ fragments; single chain Fᵥ (scFᵥ) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fᵥ fragments, scFᵥ tandems ((scFᵥ)₂ fragments), diabodies, tribodies or tetrabodies, which typically are covalently linked or otherwise stabilized (i.e., leucine zipper or helix stabilized) scFᵥ fragments. “Binding reagents” also include aptamers, as are described in the art.

Methods of making antigen-specific binding reagents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475,096, 5,840,867 and 6,544,776.

The ELISA and Luminex LabMAP immunoassays described below are examples of sandwich assays. The term “sandwich assay” refers to an immunoassay where the antigen is sandwiched between two binding reagents, which are typically antibodies. The first binding reagent/antibody being attached to a surface and the second binding reagent/antibody comprising a detectable group. Examples of detectable groups include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

In the bead-type immunoassays described in the examples below, the Luminex LabMAP system is utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantitation purposes because the bead technology does not require pre-processing or titering of the plasma or serum sample, with its inherent difficulties in reproducibility, cost and technician time. For this reason, although other immunoassays, such as, without limitation, ELISA, RIA and antibody microarray technologies, are capable of use in the context of the present invention, but they are not preferred. As used herein, “immunoassays” refer to immune assays, typically, but not exclusively sandwich assays, capable of detecting and quantifying a desired blood biomarker, namely at least one of Eotaxin, MCP-1, IL-12p40, SHBG, MMP-9, Adiponectin, Haptoglobin, FGF basic, IgM, Growth Hormone, Factor VII, or any combination of the foregoing.

Data generated from an assay to determine blood levels of one, two, three, or four or more of the biomarkers Eotaxin, MCP-1, IL-12p40, SHBG, MMP-9, Adiponectin, Haptoglobin, FGF basic, IgM, Growth Hormone, Factor VII can be used to determine the likelihood of a patient suffering from sickle cell. As shown herein, if any one or more, two or more, typically three or four or more of abnormal levels of Eotaxin, MCP-1, IL-12p40, SHBG, MMP-9, Adiponectin, Haptoglobin, FGF basic, IgM, Growth Hormone, Factor VII are present in a patient's blood, there is a very high likelihood that the patient has sickle cell. “Abnormal” levels are defined herein as any concentration above or below that which would be found or expected in a person without sickle cell. In a preferred embodiment, the “abnormal” concentration is statistically significantly different from a normal concentration. “Statistically different” is defined herein as the mean of the disease group having a p-value less than 0.05 when compared to healthy age matched controls.

In one embodiment, either an depressed Eotaxin level or a depressed Monocyte Chemotactic Protein-1 level alone, relative to the level of the biomarker of interest in a population of normal or control patients, indicates the existence of sickle cell in the patient with about a 97-99% level of certainty. (See Tables 1 and 2, discussed further below.)

In the context of the present disclosure, “blood” includes any blood fraction, for example serum, that can be analyzed according to the methods described herein. Serum is a standard blood fraction that can be tested, and is tested in the Examples below. By measuring blood levels of a particular biomarker, it is meant that any appropriate blood fraction can be tested to determine blood levels and that data can be reported as a value present in that fraction.

Collection and storage of blood specimens: Blood spots are collected. Dried blood spot specimens are clinical specimens collected by carefully applying a few drops of blood, freshly drawn by finger stick with a lancet from adults, or by heel stick with a lancet from infants, onto specially manufactured absorbent specimen collection (filter) paper. The blood is allowed to thoroughly saturate the paper and is air dried for a minimum of 3 hours. Caked or clotted specimens are not acceptable and are therefore not shipped. The specimen collection technique and the specifications for specimen matrix and shipment have been published as a national standard by the National Committee for Clinical Laboratory Standards.

Specimen collection materials (“collection kits”) for newborn screening may include a sturdy paper overlay that covers the absorbent filter paper containing the dried specimen. These are then enclosed and sealed in a highquality bond envelope. The paper overlay and the sealed bond envelope provide a double-layer barrier that protects casual handlers (i.e., shipping handlers and other nonlaboratory, non-technical personnel) from accidental exposure to the dried blood specimens and protects the specimens from exposure to the environment during shipping.

The dried blood spot specimens should not be packaged in airtight, leak-proof plastic bags because the lack of air exchange in the inner environment of a sealed plastic bag causes heat buildup and moisture accumulation that can damage the dried blood spot test substances. In addition, various chemicals that can adversely affect the test substances in the dried blood spots could leach from these plastics and thus cause incorrect analytical test results. The inclusion of desiccant packs will aid in prevention of moisture accumulation, but shipping conditions are uncontrolled and desiccant has a limited effectiveness.

Dried blood spot specimens are mailed to laboratories where the specimens are subject to various analytical procedures. One of the most important uses of this specimen collection technique is in screening for congenital and inherited metabolic disorders among the more than 4.2 million infants born annually in the United States. Efficient collection, shipment, and analysis of dried blood spot specimens on filter paper comprise the foundation of this important public health service. Other important applications include DNA (genetic) analyses, forensic studies, immunologic studies, and nutritional evaluations of infants, children, and adults.

Development of Luminex assay. The reagents for multiplex system were developed using antibody pairs purchased from R&D Systems (Minneapolis, Minn.), Fitzgerald Industries International (Concord, Mass.) or produced by well known immunological methods. Capture antibodies were monoclonal and detection antibodies were polyclonal. Capture Abs were covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μL.

This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μg of antibody. The mixture was incubated overnight in the dark with continuous shaking. Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4 C.

Coupling efficiency of monoclonal antibodies was tested by staining 2,000 microspheres with PE-conjugated goat anti-mouse IgG (BD Biosciences, San Diego, Calif.). Detection Abs were biotinylated using EZ-Link Sulfo-NHS-Biotinylation Kit (Pierce, Rockford, Ill.) according to manufacturer's protocol. The extent of biotin incorporation was determined using HABA assay and was 20 moles of biotin per mole of protein. The assays were further optimized for concentration of detection Ab and for incubation times. Sensitivity of the newly developed assays were determined using serially diluted purified proteins. Intra-assay variability, expressed as a coefficient of variation, was calculated based on the average for patient samples and measured twice at two different time points. The intra-assay variability within the replicates is expressed as an average coefficient of variation. Inter-assay variability was evaluated by testing quadruplicates of each standard and sample with an average of 16.5%. Newly developed kits were multiplexed together and the absence of cross-reactivity was confirmed according to Luminex protocol.

Statistical Analysis of Data. All statistical analyses were conducted using S-Plus statistical software (Seattle, Wash.: Math Soft, Inc., 1999). The data were first randomly split into a training and test set; described in Table C. Logistic regression (Hosmer, D W, S Lemeshow, Applied Logistic Regression. New York, N.Y.: John Wiley & Sons, 1989) was then used to calculate the optimal weighting of each biomarker and the subsequent predicted probability of being a case. All predicted probabilities ≧0.5 were categorized as a predicted case; predicted probabilities <0.5 were categorized as a predicted control. After fitting a logistic model to the training set, classification of disease status was then calculated for the test set.

### Example I

Patient Population. The patient population was chosen based on conclusive diagnosis of SCD. A sample of blood, which was tested, was obtained on admission to the hospital. The normal or control patient population was chosen from a wellness clinic. These control patients had no indication of suffering from sickle cell. Consent and blood specimens from all participants were obtained under IRB Protocol.

Collection and storage of blood specimens: Ten mL of peripheral blood was drawn from subjects using standardized phlebotomy procedures. Blood samples were collected without anticoagulant into two 5 mL red top vacutainers, sera were separated by centrifugation, and all specimens were immediately frozen and stored in the dedicated −80 C freezer. All blood samples were logged on the study computer to track information such as storage date, freeze/thaw cycles and distribution.

Additionally, CA-125 reagent for multiplex system was developed using antibody pair purchased from Fitzgerald Industries International (Concord, Mass.). Capture antibody was monoclonal and detection antibody was sheep polyclonal. Capture Ab was biotinylated using EZ-Link Sulfo-NHS-Biotinylation Kit (Pierce, Rockford, Ill.) according to the manufacturer's protocol. The extent of biotin incorporation was determined using HABA assay and was 20 moles of biotin per mole of protein. Capture Ab was covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μL. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μg of antibody. The mixture was incubated overnight in the dark with continuous shaking. Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4 C. Coupling efficiency of monoclonal antibodies was tested by staining 2,000 microspheres with PE-conjugated goat anti-mouse IgG. The assay was further optimized for concentration of detection Ab and for incubation times. Sensitivity of the newly developed assay as determined in a Luminex assay using serially diluted purified CA-125, was 20 IU. Intra-assay variability, expressed as a coefficient of variation, was calculated based on the average for patient samples and measured twice at least two different time points.

The intra-assay variability within the replicates presented as an average coefficient of variation was 8.5%. Interassay variability was evaluated by testing quadruplicates of each standard and 10 samples. The variabilities of these samples were between 10 and 22%, with an average of 16.5%. Next, the anti-CA-125 microspheres were combined with the existing multiplex kit.

Statistical Analysis of Data. All statistical analyses were conducted using S-Plus™ statistical software (Seattle, Wash.: Math Soft, Inc., 1999). The data were first randomly split into a training and test set. Logistic regression (Hosmer, D W, S Lemeshow, Applied Logistic Regression. New York, N.Y.: John Wiley & Sons, 1989) was then used to calculate the optimal weighting of each biomarker and the subsequent predicted probability of being a case. All predicted probabilities ≧0.5 were categorized as a predicted case; predicted probabilities <0.5 were categorized as a predicted control. After fitting a logistic model to the training set, classification of disease status was then calculated for the test set.

### RESULTS

Serum concentrations of biomarkers by LabMap technology. Circulating concentrations of different serum biomarkers were evaluated in a multiplexed assay using LabMap technology in blood of patients from affected and control groups. Table 1 lists the analytes that are statistically different between the two groups.

Table 2 illustrates the diagnostic accuracy obtained by testing for each individual analyte and determining how useful it would be as a diagnostic tool.

TABLE 2
Analyte Accuracy for HGB FA vs HGB SS

| | Neg | Pos | Accuracy |
|---|---|---|---|
| Eotaxin | | | |
| True Neg | 10 | 0 | |
| True Pos | 0 | 10 | 100% |
| MCP-1 | | | |
| True Neg | 8 | 2 | |
| True Pos | 2 | 8 | 80% |
| IL-12p40 | | | |
| True Neg | 10 | 0 | |
| True Pos | 2 | 8 | 90% |
| SHBG | | | |
| True Neg | 8 | 2 | |
| True Pos | 2 | 8 | 80% |
| Factor VII | | | |
| True Neg | 8 | 2 | |
| True Pos | 2 | 8 | 91% |
| MMP-9 | | | |
| True Neg | 8 | 2 | |
| True Pos | 2 | 8 | 80% |
| Adiponectin | | | |
| True Neg | 7 | 3 | |
| True Pos | 3 | 7 | 88% |
| Haptoglobin | | | |
| True Neg | 9 | 1 | |
| True Pos | 2 | 8 | 85% |
| FGF-basic | | | |
| True Neg | 7 | 3 | |
| True Pos | 1 | 9 | 80% |
| IgM | | | |
| True Neg | 6 | 4 | |
| True Pos | 1 | 9 | 75% |
| Growth | | | |
| Factor | | | |
| True Neg | 7 | 3 | |
| True Pos | 0 | 10 | 85% |
| Factor VII | | | |
| True Neg | 6 | 4 | |
| True Pos | 9 | 1 | 75% |

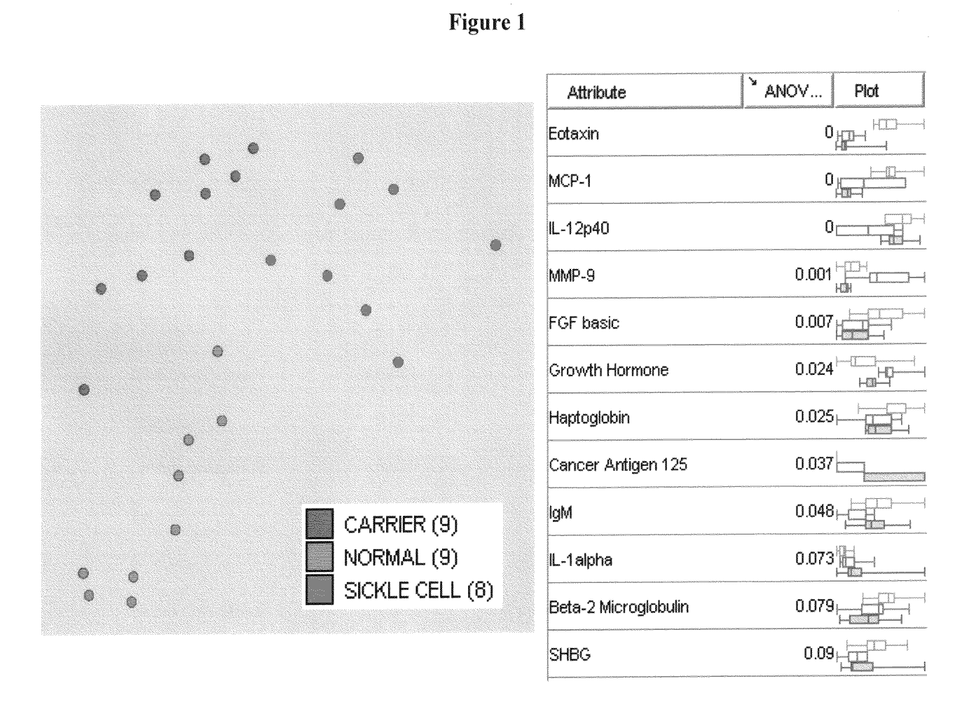

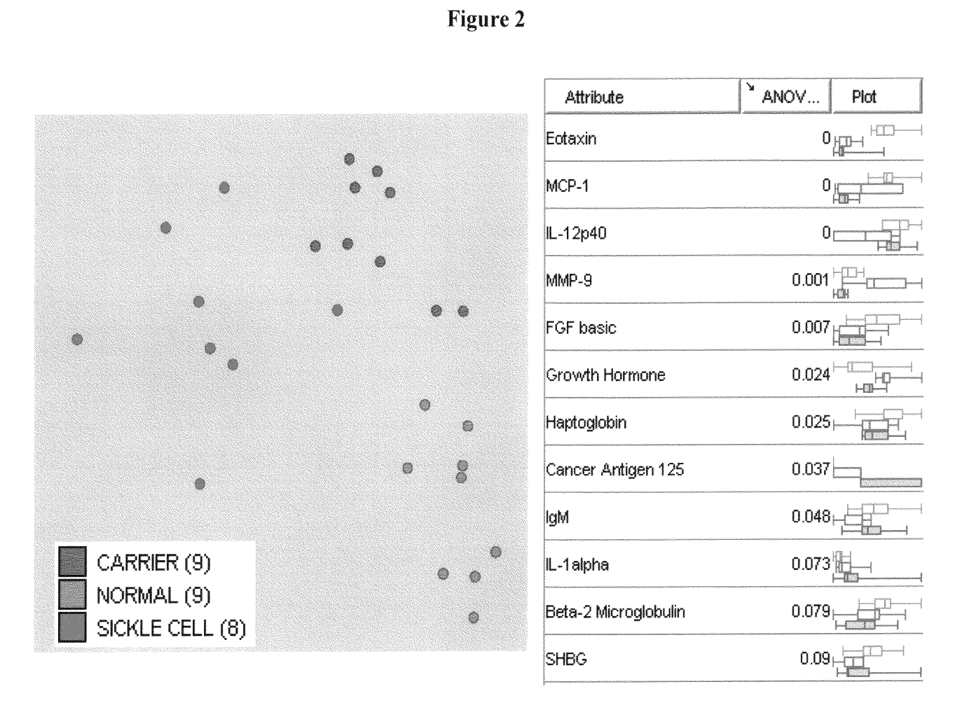

Proximity Map Analysis. The proximity map data analysis (as shown in FIGS. 1 and 2) is conducted with a software program that groups samples by their similarities in analyte concentration patterns. A unique chemical signature is generated using the concentration of the analytes measured in each sample. The relationship of each sample signature is visualized in the Galaxy™ projection. The Galaxy™ is a proximity map, such that the closer two objects are in the visualization, the closer their chemical signatures are, and thus the more similar they are to one another. The axes are dimensionless (a result of being derived from a principal component analysis), and thus the visualization is not a typical X-Y scatter plot in which moving along one axis means increasing or decreasing a single value. The two axes of the Galaxy™ are defined by the first two principal components, a common method to reduce complex data. The placement of objects (record points) is done using a set of heuristics that have been designed to maximize the preservation of spatial relationships that existed in the high-dimensional space of the original data while minimizing the overlap that can occur when doing simple projections.

What is claimed is:

1. A method of diagnosing sickle cell in a human subject suspected of suffering from sickle cell, comprising:
(a) obtaining a fluid sample from a human subject suspected of suffering from sickle cell;
(b) determining the concentration of Eotaxin in said fluid sample;
(c) deciding if the determined concentration of Eotaxin in said fluid sample is statistically different from that found in a control group of human subjects,
whereby a statistically different depressed concentration of Eotaxin supports a positive diagnosis of sickle cell.

2. The method of claim 1 in which said fluid sample is selected from the group consisting of whole blood, plasma, serum, or urine.

3. The method of claim 1 in which a determined concentration of about 300 pg/mL of Eotaxin in said fluid sample supports a positive diagnosis.

4. A method of diagnosing sickle cell in a human subject suspected of suffering from sickle cell, comprising:
(a) obtaining a fluid sample from a human subject suspected of suffering from sickle cell;
(b) determining the concentration of Monocyte Chemotactic Protein-1 in said fluid sample;
(c) deciding if the determined concentration of Monocyte Chemotactic Protein-1 in said fluid sample is statistically different from that found in a control group of human subjects,
whereby a statistically different depressed concentration of Monocyte Chemotactic Protein-1 supports a positive diagnosis of sickle cell.

5. The method of claim 4 in which said fluid sample is selected from the group consisting of whole blood, plasma, serum, or urine.

6. The method of claim 5 in which a determined concentration of 2000 pg/mL of Monocyte Chemotactic Protein-1 in said fluid sample supports a positive diagnosis.

7. A method of diagnosing sickle cell in a human subject suspected of suffering from sickle cell, comprising:
(a) obtaining a fluid sample from a human subject suspected of suffering from sickle cell;
(b) determining the concentrations of Eotaxin and Monocyte Chemotactic Protein-1 in said fluid sample;
(c) deciding if the determined concentrations of Eotaxin and Monocyte Chemotactic Protein-1 in said fluid sample are statistically different from that found in a control group of human subjects,
whereby a statistically different depressed concentration of MCP-1 and a statistically different depressed concentration of Eotaxin support a positive diagnosis of sickle cell.

8. The method of claim 7 further comprising determining the concentration in said fluid sample of at least one of IL-12p40, SHBG, MMP-9, Adiponectin, Haptoglobin, FGF basic, IgM, Growth Hormone, Factor VII, or any combination thereof.

9. The method of claim 10 in which statistically abnormal concentrations, compared to control levels, of all analytes except Monocyte Chemotactic Protein-1 support a positive diagnosis of sickle cell.

10. The method of claim 9 in which concentrations are determined by conducting one or more immunoassays.

11. The method of claim 9 further comprising determining the concentration in said fluid sample of at least one of IL-12p40, SHBG, MMP-9, Adiponectin, Haptoglobin, FGF basic, IgM, Growth Hormone, Factor VII, or any combination thereof.

12. The method of claim 11 in which statistically abnormal concentrations, compared to control levels, of all analytes except Monocyte Chemotactic Protein-1 support a positive diagnosis of sickle cell.

13. The method of claim 9 in which a subject's determined concentrations of analytes in said fluid sample are presented in a proximity map, whereby the proximity of a subject's determined concentrations to a cluster of other subjects' determined concentrations, who were previously diagnosed as having suffered from sickle cell, contributes to a positive diagnosis of sickle cell.

14. The method of claim 9 which includes applying a statistical method selected from the group consisting of linear regression analysis, classification tree analysis and heuristic nave Bayes analysis.

15. The method of claim 10 in which determined concentrations of one or more of the following analytes in said fluid sample supports a positive diagnosis of sickle cell: Eotaxin, MCP-1, IL-12p40, SHBG, MMP-9, Adiponectin, Haptoglobin, FGF basic, IgM, Growth Hormone, Factor VII or any combination thereof.

16. A kit comprising reagents for determining the concentration in a fluid sample of a panel of analytes including Eotaxin, Monocyte Chemotactic Protein-1 and one or more of IL-12p40, SHBG, MMP-9, Adiponectin, Haptoglobin, FGF basic, IgM, Growth Hormone, Factor VII.

17. The kit of claim 16 which includes antibodies against a panel of analytes including Eotaxin, MCP-1, IL-12p40, SHBG, MMP-9, Adiponectin, Haptoglobin, FGF basic, IgM, Growth Hormone, Factor VII.

18. The kit of claim 16 which includes reagents immobilized on a substrate.

19. The kit of claim 16 which the substrate comprises a two-dimensional array, a microtiter plate, or multiple bead sets.
